(19) United States
(12) Patent Application Publication
(10) Pub. No.: US 2008/0161407 A1
(43) Pub. Date: Jul. 3, 2008

(54) METHODS FOR TREATING METABOLIC SYNDROME

(75) Inventors: Kristen M. Beckman, Park Ridge, IL (US); Thomas P. McDonnell, North Wales, PA (US); Cheryl L. Renz, Kildeer, IL (US)

Correspondence Address: PAUL D. YASGER;ABBOTT LABORATORIES, 100 ABBOTT PARK ROAD, DEPT. 377/AP6A, ABBOTT PARK, IL, 60064-6008, US

(73) Assignee: Abbott Laboratories, Abbott Park, IL (US)

(21) Appl. No.: 12/048,687

(22) Filed: Mar. 14, 2008

Related U.S. Application Data
(63) Continuation of application No. 10/911,086, filed on Aug. 4, 2004, now abandoned.
(60) Provisional application No. 60/492,350, filed on Aug. 4, 2003.

(51) Int. Cl. A61K 31/135; A61P 3/04; A61P 3/06; A61P 3/08

(52) U.S. Cl. 514/646

(57) ABSTRACT

The invention pertains, at least in part, to a method for treating metabolic syndrome in a subject. The method includes administering to a subject an effective amount of a compound of formula (I). The compound of formula (I) is:

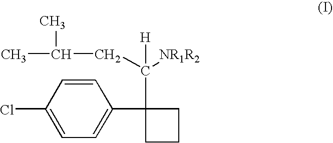

wherein R₁ and R₂ are independently H or methyl, and enantiomers and pharmaceutically acceptable salts thereof.

### CROSS REFERENCE TO RELATED APPLICATION

This application is a continuation of U.S. application Ser. No. 10/911,086, filed on Aug. 4, 2004, which claims priority to U.S. Provisional Application No. 60/492,350, filed Aug. 4, 2003, both of which are hereby incorporated by reference in its entirety.

### BACKGROUND OF THE INVENTION

Metabolic syndrome is associated with a constellation of metabolic abnormalities which are believed to be associated with insulin resistance or impaired glucose tolerance (Hansen, B C (1999) Ann NY Acad Sci 892:1). Metabolic syndrome has been recognized as a combination of three or more of the following: abdominal obesity, elevated triglyceride levels, decreased high-density lipoprotein (HDL) cholesterol levels, high blood pressure, and impaired fasting blood glucose, (a measure for decreased insulin sensitivity and increased risk of developing diabetes). (Expert panel on detection evaluation and treatment of high blood cholesterol in adults (2001) JAMA 285:2486-2497). Metabolic syndrome significantly increases the risk of coronary heart disease (CHD) and atherogenesis, and has been identified as an independent target for coronary heart disease risk reduction, separate from low-density lipoprotein (LDL) cholesterol elevations. Metabolic syndrome is highly prevalent, with estimates as high as one in four US adults (Ford, E S, et. al. (2002) JAMA 287:356-359).

One abnormality of metabolic syndrome is abdominal obesity, or accumulated visceral fat (Maison, P, et. al. (2001) Diabetes Care 24:1758-1763; DePres, J-P, et. al., (2001) BMJ 322:716-720). Visceral fat has unique endocrine and metabolic properties that differentiate it from other fat deposits in the body (i.e. subcutaneous fat), and negatively impact glucose and lipid metabolism (Grundy, S M (2000) Endocrine 13:155-165). Accumulations in visceral fat are directly correlated to increased insulin resistance, dyslipidemia, hypertension, and coronary heart disease (Pouliot, M-C, et al (1994) 73:460-468). Visceral fat is most accurately measured by radiographic techniques like computed tomography, but is closely correlated to waist circumference, which can be easily measured and followed by clinicians.

Weight reduction and increased physical activity have been recommended as the first line therapy for metabolic syndrome. Weight control and physical activity have been shown to independently benefit lipid and non-lipid metabolic risk factors, and are associated with reductions in morbidity and mortality. Even moderate weight reductions of 5-10% have significant clinical benefit. Although safe and effective pharmacotherapy exists for individual metabolic syndrome components, a specific drug therapy for metabolic syndrome as a whole has not been established.

### SUMMARY OF THE INVENTION

In an embodiment, the invention pertains to a method for treating metabolic syndrome in a subject. The method includes preselecting a subject suffering from metabolic syndrome, and administering to the subject an effective amount of a compound of formula (I), such that the subject is treated for the metabolic syndrome. The compound of formula (I) is:

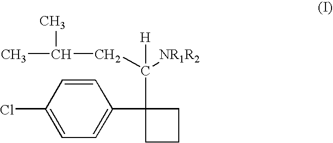

wherein R₁ and R₂ are independently H or methyl, and enantiomers and pharmaceutically acceptable salts thereof.

In one embodiment, the invention pertains, at least in part, to a method for treating metabolic syndrome in a subject. The method includes administering to the subject an effective amount of a compound of formula (I), such that the subject is treated.

In another embodiment, the invention pertains to a method for treating metabolic syndrome in a subject. The method includes preselecting a subject suffering from three or more symptoms of metabolic syndrome. The method includes administering to the subject an effective amount of a compound of formula (I), such that the subject is treated for said metabolic syndrome.

In another embodiment, the invention pertains, at least in part, to a packaged pharmaceutical composition, which is comprised of an effective amount of a compound of formula (I) for the treatment of metabolic syndrome packaged together with directions for using said compound to treat metabolic syndrome.

### DETAILED DESCRIPTION OF THE INVENTION

In one embodiment, the invention pertains, at least in part, to methods for treating metabolic syndrome in a subject. The method includes administering to the subject an effective amount of a compound of formula (I), such that the subject is treated for metabolic syndrome. The compound of formula (I) is:

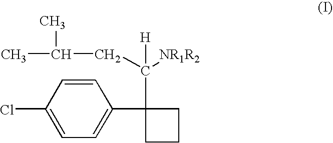

wherein R₁ and R₂ are independently H or methyl, and enantiomers and pharmaceutically acceptable salts thereof. Compounds of formula (I) may also be referred to herein as sibutramine compounds. Compounds wherein R₁ and R₂ are each methyl may also be referred to herein as sibutramine. Compounds wherein R₁ is hydrogen and R₂ is methyl or wherein R₁ and R₂ are each hydrogen may be referred to as sibutramine metabolites. Methods for using the sibutramine compounds to treat metabolic disorders may also be referred to as “sibutramine therapy.”

Sibutramine has been marketed and promoted to produce clinically significant weight loss in obese patients and overweight patients with cardiovascular risk factors; the weight loss observed in subjects treated with sibutramine or sibutramine metabolites is associated with improvements in metabolic risk factors that characterize metabolic syndrome, e.g., decreased abdominal obesity, improved high density lipoprotein (HDL) and triglyceride lipid profiles.

The methods of the invention are effective for subjects with metabolic syndrome. The methods of the invention are uniquely effective for metabolic syndrome because a compound of formula (I) of the invention positively impact multiple abnormalities of the syndrome. In this way, the methods of the invention differ from other treatments that target individual metabolic disorders like dyslipidemia or hypertension.

Clinical trials have demonstrated significant improvements in HDL cholesterol, triglyceride levels, waist circumference (a measure for abdominal obesity), and insulin levels in patients who take sibutramine compared to placebo, (Astrup, A, et. al. (2001) Int J Obes Relat Metab Disord 24 (suppl. 2):S104; James, W P T, et. al. (2000) Lancet 356:2119-2125). The effects of sibutramine on impaired fasting blood glucose have not been assessed prospectively, but the decrease in insulin levels associated with sibutramine treatment would suggest an improvement in insulin sensitivity; fasting blood glucose levels are not significantly changed by sibutramine when compared to placebo. The changes associated with sibutramine therapy on abdominal obesity and its associated metabolic risk factors predict lower risks of heart disease, stroke, and the onset of diabetes.

Sibutramine therapy has demonstrated efficacy in improving HDL and triglyceride dyslipidemias, abdominal adiposity, and has a positive effect on insulin resistance. In addition, weight loss and maintenance positively affects all abnormalities of metabolic syndrome, and is considered first-line therapy.

The metabolic derangements associated with obesity have been identified previously, but only recently has metabolic syndrome been formally defined and identified as a condition that incurs increased risk for cardiovascular disease, diabetes, and mortality. Treatment guidelines for metabolic syndrome focus on weight control and increased physical activity. Weight loss and increased activity are the only intervention to date that is indicated for all of the metabolic syndrome components. Pharmacologic therapy for subjects with metabolic syndrome has otherwise been disease-specific, i.e. anti-lipid therapy for dyslipidemia and anti-hypertensive therapy for elevated blood pressure. Recently, weight control and increased physical activity has also been identified as the treatment of choice to delay the onset of diabetes mellitus in patients with glucose intolerance (Tuomilehto, J, et. al. (2001) 344:1343-1350; Diabetes Prevention. Research Group (2002) N Engl J Med 346:393-403).

Treatment using a compound of formula (I) of the invention is uniquely effective for metabolic syndrome because it positively impacts multiple abnormalities of the syndrome. Clinical trials have demonstrated significant improvements in HDL cholesterol, triglyceride levels, waist circumference (a measure for abdominal obesity), and insulin levels (an indicator of insulin resistance), in subjects who take a compound of formula (I) of the invention compared to placebo (James, W P T, et. al. (2000) Lancet 356:2119-2125). In this way, treatment with a compound of formula (I) of the invention differs from other treatments that target individual metabolic disorders like dyslipidemia, hypertension, or impaired glucose tolerance. The benefits of weight loss and maintenance is extended to those subjects who are unable to achieve weight control by diet and exercise alone.

In an embodiment, a compound of formula (I) of the invention can be used to treat patients with metabolic syndrome in conjunction with a low calorie diet and a program of increased physical activity.

The term “metabolic syndrome” includes a combination of three or more of the following symptoms: abdominal obesity, elevated triglyceride levels, decreased high-density lipoprotein (HDL) cholesterol levels, high blood pressure, and impaired fasting blood glucose (a measure for decreased insulin sensitivity and increased risk of developing diabetes).

The term “treat” includes the amelioration or reduction of three or more of the symptoms of metabolic syndrome. For example, treating metabolic syndrome in a subject may result in a reduction of abdominal obesity, lowering of elevated triglyceride levels, and increasing high-density lipoprotein cholesterol levels. In another example, treating metabolic syndrome in a subject may result in improved fasting blood glucose levels, lowered blood pressure, and increased HDL cholesterol levels. Other combinations of beneficially affecting the symptoms of metabolic syndrome are also included.

In one embodiment, the effective amount of a compound of formula (I) is effective to reduce triglyceride levels in the subject. In a further embodiment, a compound of formula (I) is effective to reduce triglyceride levels in the subject by 5% or greater, by 10% or greater, by 15% or greater, by 20% or greater, by 25% or greater, by 30% or greater, by 35% or greater, by 40% or greater, by 45% or greater, by 50% or greater, by 55% or greater, by 60% or greater, by 65% or greater, by 70% or greater, by 75% or greater, by 80% or greater, by 85% or greater, by 90% or greater or by 95% or greater. Preferably the reduction in triglyceride level in the subject is at least 19%.

In another embodiment, the effective amount of a compound of formula (I) is effective to increase high-density lipoprotein cholesterol levels in the subject. In a further embodiment, the effective amount is effective to increase HDL cholesterol levels by 5% or greater, by 10% or greater, by 15% or greater, by 20% or greater, by 25% or greater, by 30% or greater, by 35% or greater, by 40% or greater, by 45% or greater or by 50% or greater. Preferably the increase in HDL levels in the subject is at least 15%.

In another embodiment, the effective amount of a compound of formula (I) is effective to treat impaired fasting blood glucose. In a further embodiment, the subject's fasting blood glucose is modulated to a level considered to be healthy for said subject's age, weight and activity level.

In another embodiment, the effective amount of a compound of formula (I) is effective to decrease abdominal obesity in a subject. In one embodiment, the subject's waist circumference is decreased by one inch or greater, 2 inches or greater, 3 inches or greater, 4 inches or greater, 5 inches or greater, 6 inches or greater, 7 inches or greater, 8 inches or greater, 9 inches or greater, 10 inches or greater; 11 inches or greater, 12 inches or greater, 13 inches or greater, 14 inches or greater, 15 inches or greater, 16 inches or greater, 20 inches or greater, 24 inches or greater, 28 inches or greater, 32 inches or greater, 36 inches or greater, or 40 inches or greater. In another embodiment, the subject's waist circumference is decreased by 5% or greater, 10% or greater, 15% or greater, 20% or greater, 25% or greater, 30% or greater, 35% or greater, 40% or greater, 45% or greater, or 50% or greater. Preferably the subject's waist circumference is decreased at least 8 inches.

The term “subject” includes animals which are capable of suffering from metabolic syndrome. Examples of subjects include horses, rats, rabbits, mice, cows, pigs, bears, dogs, cats, ferrets, rabbits, etc. In a preferred embodiment, the subject is a primate, preferably a human. In a further embodiment, the subject has a BMI (Body Mass Index) of 27 or greater, 28 or greater, 29 or greater, 30 or greater, 31 or greater, 32 or greater, 33 or greater, 34 or greater, 35 or greater, 36 or greater, 37 or greater, 38 or greater, 39 or greater, 40 or greater, 42 or greater, 44 or greater, 46 or greater, 46 or greater, or 48 or greater.

In an embodiment, the invention pertains to a method for treating metabolic syndrome in a subject. The method includes preselecting a subject suffering from metabolic syndrome, and administering to the subject an effective amount of a compound of formula (I), such that the subject is treated for the metabolic syndrome.

The term “preselecting” includes screening and other methods for identifying subjects suffering from metabolic syndrome or a particular combination of two, three, four or five symptoms of metabolic syndrome. The preselection of subjects may be done by physicians treating particular subjects suffering from the combination of symptoms, or it may be done by marketing the drug to persons suffering from the particular combination of symptoms or their health care providers.

In another embodiment, the invention pertains to a method for treating metabolic syndrome in a subject. The method includes preselecting a subject suffering from two, three, four, five or more symptoms of metabolic syndrome. The method includes administering to the subject an effective amount of a compound of formula (I), such that the subject is treated for said metabolic syndrome. Examples of symptoms include abdominal obesity, elevated triglyceride levels, decreased high-density lipoprotein (HDL) cholesterol levels, high blood pressure, and impaired fasting blood glucose.

In a further embodiment, the invention pertains to methods for treating metabolic syndrome by administering a compound of formula (I) in combination with a second agent.

The language “in combination with” a second agent or treatment includes co-administration of a compound of formula (I), and with the second agent or treatment, administration of a compound of formula (I) first, followed by the second agent or treatment and administration of the second agent or treatment first, followed by a compound of formula (I). The second agent may be any agent which is known in the art to treat, prevent, or reduce a symptom of metabolic syndrome, e.g., reduce high blood pressure, aid in appetite control, etc. Furthermore, the second agent may be any agent of benefit to the patient when administered in combination with the administration of a compound of formula (I).

### Compounds of the Invention and Preparation Thereof

In one embodiment, the compounds of the invention are of formula (I):

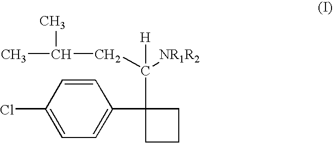

wherein R₁ and R₂ are independently H or methyl, and enantiomers and pharmaceutically acceptable salts thereof.

Sibutramine (Formula I, R₁=CH₃, R₂=CH₃) has a pharmacological profile which is unique amongst monoamine reuptake inhibitors. Through its pharmacologically active metabolites,: (metabolite 1, R₁=H, R₂=CH₃ in Formula I and metabolite 2, R₁=H, R₂=H in Formula I) sibutramine inhibits the reuptake of all three monoamines differentiating it from serotonin (5-HT)-selective reuptake inhibitors, e.g. fluoxetine, noradrenaline-selective reuptake inhibitors, e.g. desipramine, dopamine-selective reuptake inhibitors, e.g. bupropion, and serotonin-noradrenaline reuptake inhibitors, e.g. venlafaxine. It is this unique combination of pharmacological actions which renders sibutramine, and the other compounds of formula I, efficacious in the treatment of metabolic syndrome.

Sibutramine is a serotonin and noradrenaline reuptake inhibitor that acts centrally to reduce energy intake by inducing a feeling of fullness (or satiety) after eating and affecting energy expenditure. Treatment with sibutramine leads to reduced food intake and a decreased tendency toward snacking. Sibutramine does not induce anorexia (loss of appetite). With weight loss, there is normally a decline in metabolic rate; however, sibutramine limits the decline in metabolic rate that normally accompanies weight loss.

Compounds of formula I contain a chiral center. When a compound of formula I contains a single chiral centre it may exist in two enantiomeric forms. The present invention includes the use of the individual enantiomers and mixtures of the enantiomers. The enantiomers may be resolved by methods known to those skilled in the art, for example by formation of diastereoisomeric salts or complexes which may be separated, for example, by crystallization; via formation of diastereoisomeric derivatives which may be separated, for example, by crystallization, gas-liquid or liquid chromatography; selective reaction of one enantiomer with an enantiomer-specific reagent, for example enzymatic oxidation or reduction, followed by separation of the modified and unmodified enantiomers; or gas-liquid or liquid chromatography in a chiral environment, for example on a chiral support, for example silica with a bound chiral ligand or in the presence of a chiral solvent. It will be appreciated that where the desired enantiomer is converted into another chemical entity by one of the separation procedures described above, a further step is required to liberate the desired enantiomeric form. Alternatively, specific enantiomers may be synthesized by asymmetric synthesis using optically active reagents, substrates, catalysts or solvents, or by converting one enantiomer to the other by asymmetric transformation.

The individual enantiomers can be prepared by enantioselective synthesis from optically active precursors, or by resolving the racemic compound which can be prepared as described above. Enantiomers of secondary amines of the formula I can also be prepared by preparing the racemate of the corresponding primary amine, resolving the latter into the individual enantiomers, and then converting the optically pure primary amine enantiomer into the required secondary amine by methods described in British Patent Specification 2098602. Specific examples of compounds of formula I are: (+)-N-[1-[1-(4-chlorophenyl)cyclobutyl]-3-methylbutyl}-N-methylamine; (−)-N-{1-[1-(4-chlorophenyl)cyclobutyl-3-methylbutyl}-N-methylamine; (+)-1-[1-(4-chlorophenyl)cyclobutyl]-3-methylbutylamine; (−)-1-[1-(4-chlorophenyl)cyclobutyl]-3-methylbutylamine; (+)-N-{1-[1-(4-chlorophenyl)cyclobutyl]-3-methylbutyl}-N,N-dimethylamine; (−)-N-{1-[1-(4-chlorophenyl)cyclobutyl]-3-methylbutyl}-N,N-dimethylamine; (+/−)-N-{1-[1-(4-chlorophenyl)cyclobutyl}-N-methylamine; (+/−)-1-[1-(4-chlorophenyl)cyclobutyl]-3-methylbutylamine; (+/−)-N-{1-[1-(4-chlorophenyl)cyclobutyl]-3-methylbutyl}-N,N-dimethylamine, and mixtures, and pharmaceutically acceptable salts thereof.

The preparation of compounds of formula I, such as N,N-dimethyl-1-[1-(4-chlorophenyl)cyclobutyl]-3-methylbutylamine and salts thereof is described in British Patent Specification 2098602 and U.S. Pat. No. 4,522,828. The use of a compound of formula I, and salts thereof in the treatment of the following conditions and diseases are described as noted: treating Parkinson's disease is described in WO 88/06444; treating cerebral function disorders is described in U.S. Pat. No. 4,939,175; treating obesity is described in U.S. Pat. No. 5,436,272; for improving the glucose tolerance of humans having Impaired Glucose Tolerance or Non-Insulin Dependent Diabetes Mellitus is described in U.S. Pat. Nos. 5,459,164 and 5,942,549; treating depression is described in U.S. Pat. No. 6,552,087; lessening weight gain associated with certain drug therapy is described in WO 00/56313; treating chronic fatigue syndrome is described in U.S. Pat. No. 6,376,551; treating disorders arising from drug misuse is described in WO 00/56148; treating pulmonary hypertension is described in U.S. Pat. No. 6,403,650; treating menstrual dysfunction is described in U.S. Pat. No. 6,372,797; treating premenstrual syndrome is described in WO 00/56150; decreasing platelet adhesiveness is described in U.S. Pat. No. 6,380,260; treating eating disorders is described in U.S. Pat. No. 6,365,633; treating cancers is described in U.S. Pat. No. WO 00/56323; treating osteoarthritis is described in U.S. Pat. No. 6,232,347; treating cardiovascular disease is described in U.S. Pat. No. 6,433,020; treating gallstones is described in U.S. Pat. No. 6,376,552; treating neuropathic pain is described in WO 00/56318; treating obsessive-compulsive is described in WO 00/56151; treating orthostatic hypotension is described in U.S. Pat. No. 6,365,632; treating hyperactivity disorders is described in U.S. Pat. No. 6,372,798; treating sexual dysfunction is described in U.S. Pat. No. 6,376,554; treating pain is described in U.S. Pat. No. 6,376,553; in a treatment program to cease smoking is described in WO 00/43002; treating hiatial hernia is described in U.S. Pat. No. 6,288,125; treating anxiety disorders is described in U.S. Pat. No. 6,355,685; treating sleep apnea is described in U.S. Pat. No. 6,365,631; treating weight loss after pregnancy is described in WO 00/56317; promoting non-exercise activity thermogenesis is described in U.S. Pat. No. 6,441,046; reducing insulin resistance in individuals with increased risk of Glucose tolerance and non-insulin dependent diabetes mellitus is described in U.S. Pat. No. 6,174,925; treating urinary incontinence is described in U.S. Pat. No. 6,046,242; treating obesity by using a compound of formula I in combination with Orlistat™ is described in U.S. Pat. No. 6,403,641; treating obesity by using a compound of formula I in combination with a bulk forming agent is described in WO 01/34140; lowering uric acid levels is described in U.S. Pat. No. 6,162,831; treating co-morbid conditions associated with obesity with sibutramine and Orlistat™ is described in WO 01/00205; use to lower lipid levels is described in U.S. Pat. No. 6,187,820; and treating obesity and associated co-morbid conditions using a compound of formula I and a lipase inhibitor other than Orlistat™ is described in WO 01/00187.

In yet another embodiment, the invention pertains to the hydrochloride salts of compounds Formula I, but the free bases and other pharmaceutically acceptable salts are also suitable. Other preferred pharmaceutically acceptable salts include hydrochlorides, hydrobromides, sulfates, methanesulfonates, nitrates, maleates, acetates, citrates, fumarates tartrates [eg (+)-tartrates, (−)-tartrates or mixtures thereof including racemic mixtures], succinates, benzoates and salts with amino acids such as glutamic acid. These salts may be prepared by methods known to those skilled in the art.

In still another embodiment, the use of the monohydrate of the hydrochloride salt of a compound of formula (I) in the treatment of metabolic syndrome is preferred.

### Pharmaceutical Compositions

In a further embodiment, the invention also pertains to a packaged pharmaceutical composition. The packaged pharmaceutical composition comprises an effective amount of a compound of formula (I) for the treatment of metabolic syndrome and directions for using the compound to treat metabolic syndrome.

A compound of formula I may be administered in any of the known pharmaceutical dosage forms. The amount of the compound to be administered will depend on a number of factors including the age of the patient, the severity of the condition and the past medical history of the patient and always lies within the sound discretion of the administering physician but it is generally envisaged that the dosage of the compound to be administered will be in the range 0.1 to 50 mg preferably 1 to 30 mg per day given in one or more doses.

Oral dosage forms are one of the preferred compositions for use in the present invention and these are the known pharmaceutical forms for such administration, for example tablets, capsules, granules, syrups and aqueous or oil suspensions. The excipients used in the preparation of these compositions are the excipients known in the pharmacist's art. Tablets may be prepared from a mixture of the active compound with fillers, for example calcium phosphate; disintegrating agents, for example maize starch; lubricating agents, for example magnesium stearate; binders, for example microcrystalline cellulose or polyvinylpyrrolidone and other optional ingredients known in the art to permit tableting the mixture by known methods. The tablets may, if desired, be coated using known methods and excipients which may include enteric coating using for example hydroxypropylmethylcellulose phthalate. The tablets may be formulated in a manner known to those skilled in the art so as to give a sustained release of the compounds of the present invention. Such tablets may, if desired, be provided with enteric coatings by known methods, for example by the use of cellulose acetate phthalate. Similarly, capsules, for example hard or soft gelatin capsules, containing the active compound with or without added excipients, may be prepared by known methods and, if desired, provided with enteric coatings in a known manner. The contents of the capsule may be formulated using known methods so as to give sustained release of the active compound. The tablets and capsules may conveniently each contain 1 to 50 mg of the active compound, preferably 5 mg, 10 mg or 15 mg. Preferred doses of a compound of formula (I) is 5 mg, 10 mg or 15 mg. Preferred doses of sibutramine HCl monohydrate is 5 mg, 10 mg or 15 mg. Preferred doses of a sibutramine metabolite is 5 mg, 10 mg or 15 mg.

Other dosage forms for oral administration include, for example, aqueous suspensions containing the active compound in an aqueous medium in the presence of a non-toxic suspending agent such as sodium carboxy-methylcellulose, and oily suspensions containing a compound of the present invention in a suitable vegetable oil, for example arachis oil. The active compound may be formulated into granules with or without additional excipients. The granules may be ingested directly by the patient or they may be added to a suitable liquid carrier (for example, water) before ingestion. The granules may contain disintegrants, e.g. an effervescent couple formed from an acid and a carbonate or bicarbonate salt to facilitate dispersion in the liquid medium.

The therapeutically active compounds of formula I may be formulated into a composition which the patient retains in his mouth so that the active compound is administered through the mucosa of the mouth.

Dosage forms suitable for rectal administration are the known pharmaceutical forms for such administration, for example, suppositories with cocoa butter or polyethylene glycol bases.

Dosage forms suitable for parenteral administration are the known pharmaceutical forms for such administration, for example sterile suspensions or sterile solutions in a suitable solvent

Dosage forms for topical administration may comprise a matrix in which the pharmacologically active compounds of the present invention are dispersed so that the compounds are held in contact with the skin in order to administer the compounds transdermally. A suitable transdermal composition may be prepared by mixing the pharmaceutically active compound with a topical vehicle, such as a mineral oil, petrolatum and/or a wax, e.g. paraffin wax or beeswax, together with a potential transdermal accelerant such as dimethyl sulphoxide or propylene glycol. Alternatively the active compounds may be dispersed in a pharmaceutically acceptable cream, gel or ointment base. The amount of active compound contained in a topical formulation should be such that a therapeutically effective amount of the compound is delivered during the period of time for which the topical formulation is intended to be on the skin.

A therapeutically active compound of formula I may be formulated into a composition which is dispersed as an aerosol into the patients oral or nasal cavity. Such aerosols may be administered from a pump pack or from a pressurized pack containing a volatile propellant.

A therapeutically active compound of formula I used in the method of the present invention may also be administered by continuous infusion either from an external source, for example by intravenous infusion or from a source of the compound placed within the body. Internal sources include implanted reservoirs containing the compound to be infused which is continuously released for example by osmosis and implants which may be (a) liquid such as an oily suspension of the compound to be infused for example in the form of a very sparingly water-soluble derivative such as a dodecanoate salt or a lipophilic ester or (b) solid in the form of an implanted support, for example of a synthetic resin or waxy material, for the compound to be infused. The support may be a single body containing all the compound or a series of several bodies each containing part of the compound to be delivered. The amount of active compound present in an internal source should be such that a therapeutically effective amount of the compound is delivered over a long period of time.

In some formulations it may be beneficial to use the compounds of the present invention in the form of particles of very small size, for example as obtained by fluid energy milling.

In the compositions of the present invention the active compound may, if desired, be associated with other compatible pharmacologically active ingredients.

The invention further provides the use of compounds of formula I in the manufacture of a medicament for treating metabolic syndrome. The efficacy of compounds of formula I in treating metabolic syndrome is demonstrated using clinical trials in a relevant population set and the data presented below.

### Exemplification of the Invention

The effect of sibutramine hydrochloride monohydrate (in this example is referred to as sibutramine) on weight maintenance after weight loss was examined by the Sibutramine Trial of Obesity Reduction and Maintenance (STORM) Study Group (James, W P T, et. al. (2000) Lancet 356:2119-2125). This study was a randomized, double-blind, placebo-controlled trial designed to assess the effect of sibutramine treatment in combination with diet and exercise on the maintenance of weight loss over a two-year period. During the first six months, 605 subjects were treated with sibutramine and counseled on reduced calorie diet and increased physical activity. Of the 499 subjects who completed the first six months, 94% (467) achieved a ≧5% weight reduction, with a mean weight loss of 26 lbs. A total of 477 subjects were subsequently randomized to receive sibutramine or placebo, both in conjunction with diet and exercise counseling; subjects were then followed for another 18 months. At the end of two years of treatment, significantly more subjects treated with sibutramine than with placebo maintained ≧5% (69% vs. 44%) and ≧10% (46% vs. 21%) weight reductions; overall 43% of sibutramine-treated subjects who completed the trial maintained at least 80% of their month six weight loss at two years compared to 16% of the patients treated with placebo.

Subjects who lost and maintained weight while taking sibutramine had clinically and statistically significant improvements in triglycerides, HDL cholesterol, and waist circumference when compared to the baseline values of all subjects. Impaired fasting glucose was significantly changed, but mean fasting insulin levels were reduced at two years, suggesting overall improvement in insulin sensitivity.

The benefits of sibutramine therapy can be extended to subjects with metabolic syndrome. The treatment effect of sibutramine on subjects that had metabolic syndrome at study entry in the STORM trial and by a pooled analysis of double-blind, placebo-controlled, non-diabetic studies of at least 6 months duration were studied. Subjects were identified who had increased abdominal obesity (waist circumference), decreased HDL cholesterol, and elevated triglycerides (TG) at baseline, and who also completed at least 6 months of sibutramine treatment (pooled analysis subjects: 266 sibutramine, 134 placebo; STORM: 80 sibutramine). Since all subjects in the STORM trial received sibutramine for the first 6 months of the study, only subjects subsequently randomized to receive sibutramine for a longer duration were included. The analyses for each variable were based on change from baseline to latest assessment after month 6, up to and including month 12.

Results of the pooled analyses revealed that subjects treated with sibutramine had clinically and statistically significant improvements in weight loss, HDL, TG, and waist circumference compared to subjects receiving placebo. The STORM data also showed favorable changes in the sibutramine treatment group for these variables. Impaired fasting blood glucose was identified in only a small number of patients at baseline in the pooled analysis (26 placebo; 45 sibutramine), or STORM (10 patients, all received sibutramine), but demonstrated similar improvements in weight loss, waist circumference, and dyslipidemia in subjects who received sibutramine treatment compared to placebo.

### Equivalents

Those skilled in the art will recognize, or be able to ascertain using no more than routine experimentation, many equivalents to the specific embodiments of the invention described herein. Such equivalents are intended to be encompassed by the following claims.

The entire contents of all references, patents, and patent applications cited herein are expressly incorporated by reference.

What is claimed is:

1. A method for treating metabolic syndrome in a subject, comprising:
preselecting said subject suffering from metabolic syndrome;
administering to said subject an effective amount of a compound of formula (I), such that said subject is treated for said metabolic syndrome, wherein said compound of formula (I) is: 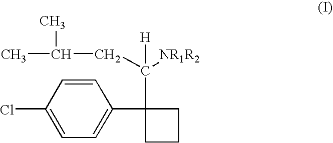
wherein R₁ and R₂ are independently H or methyl, and enantiomers and pharmaceutically acceptable salts thereof.

2. A method for treating metabolic syndrome in a subject, comprising:
administering to said subject an effective amount of a compound of formula (I), such that said subject is treated for said metabolic syndrome, wherein said compound of formula (I) is: 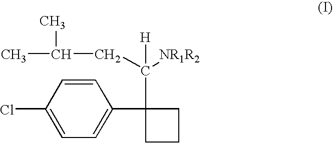
wherein R₁ and R₂ are independently H or methyl, and enantiomers and pharmaceutically acceptable salts thereof.

3. The method of claim 1, wherein R₁ is methyl and R₂ is methyl.

4. The method of claim 1, wherein R₁ is hydrogen and R₂ is methyl.

5. The method of claim 1, wherein R₁ is hydrogen and R₂ is hydrogen.

6. The method of claim 1, wherein said compound is (+)-N-[1-[1-(4-chlorophenyl)cyclobutyl]-3-methylbutyl}-N-methylamine; (+)-1-[1-(4-chlorophenyl)cyclobutyl]-3-methylbutylamine; (+)-N-{1-[1-(4-chlorophenyl)cyclobutyl]-3-methylbutyl}-N-N-dimethylamine; (−)-N-{1-[1-(4-chlorophenyl)cyclobutyl-3-methylbutyl}-N-methylamine; (−)-1-[1-(4-chlorophenyl)cyclobutyl]-3-methylbutylamine; (−)-N-{1-[1-(4-chlorophenyl)cyclobutyl]-3-methylbutyl}-N-N-dimethylamine, or mixtures thereof.

7. The method of claim 2, wherein R₁ is methyl and R₂ is methyl.

8. The method of claim 2, wherein R₁ is hydrogen and R₂ is methyl.

9. The method of claim 2, wherein R₁ is hydrogen and R₂ is hydrogen.

10. The method of claim 2, wherein said compound is (+)-N-[1-[1-(4-chlorophenyl)cyclobutyl]-3-methylbutyl}-N-methylamine; (+)-1-[1-(4-chlorophenyl)cyclobutyl]-3-methylbutylamine; (+)-N-{1-[1-(4-chlorophenyl)cyclobutyl]-3-methylbutyl}-N-N-dimethylamine; (−)-N-{1-[1-(4-chlorophenyl)cyclobutyl-3-methylbutyl}-N-methylamine; (−)-1-[1-(4-chlorophenyl)cyclobutyl]-3-methylbutylamine; (−)-N-{1-[1-(4-chlorophenyl)cyclobutyl]-3-methylbutyl}-N-N-dimethylamine, or mixtures thereof.

11. The method of claim 1, wherein said compound is administered in combination with a second therapeutic agent.

12. The method of claim 2, wherein said compound is administered in combination with a second therapeutic agent.

13. A method for treating metabolic syndrome in a subject, comprising:
preselecting said subject suffering from three or more symptoms of metabolic syndrome selected from the group consisting of abdominal obesity, elevated triglyceride levels, decreased high-density lipoprotein (HDL) cholesterol levels, high blood pressure, and impaired fasting blood glucose levels;
administering to said subject an effective amount of a compound of formula (I), such that said subject is treated for said metabolic syndrome, wherein said compound of formula (I) is: 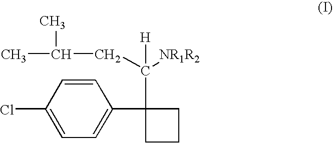
wherein R₁ and R₂ are independently H or methyl, and enantiomers and pharmaceutically acceptable salts thereof.

14. The method of claim 13, wherein said subject is suffering from four or more symptoms of metabolic syndrome selected from the group consisting of abdominal obesity, elevated triglyceride levels, decreased high-density lipoprotein (HDL) cholesterol levels, high blood pressure, and impaired fasting blood glucose levels.

15. The method of claim 13, wherein said subject is suffering from five or more symptoms of metabolic syndrome selected from the group consisting of abdominal obesity, elevated triglyceride levels, decreased high-density lipoprotein (HDL) cholesterol levels, high blood pressure, and impaired fasting blood glucose levels.

16. A packaged pharmaceutical composition, comprising an effective amount of a compound of formula (I) for the treatment of metabolic syndrome and directions for using said compound to treat metabolic syndrome, wherein said compound of formula (I) is: 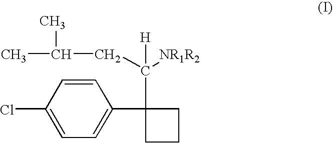
wherein R₁ and R₂ are independently H or methyl, and enantiomers and pharmaceutically acceptable salts thereof.

17. The method of claim 1, wherein the subject is human.

18. The method of claim 2, wherein the subject is human.

19. The method of claim 13, wherein R₁ and R₂ are each methyl and the. compound formula (I) is a hydrochloride salt.

20. The packaged pharmaceutical composition of claim 16 wherein R₁ and R₂ are each methyl and the compound of formula (I) is a hydrochloride salt.
